SEQUENCE LISTING (18 sequences)

SEQ ID NO: 1 - protein, 359 aa, Unknown
  Description of Unknown: Neurospora crassa ATCC 24698 sequence
  3-dehydroshikimate dehydratase
MPSKLAISSMSLGRCFAGHSLDSKLDAAQRYGYLGIELFYEDLVDVAEHLSNERPSPEGPFVEAQIAAARHILQMCQARGLEVVCLQPFMHYDGLNDRAEHERRLEKLALWIELAHELHTDIIQIPANFLPANQVSDNLDLIVSDLCKVADIGAQALPPIRFAYESLCWSTRVDLWERCWDIVQRVDRPNFGICLDTFNILGRIYADPTSPSGRTPNAKEAVRKSIANLVSRVDVSKVFYVQVVDAERLSKPLLPGHPYYNPEQPARMSWSRNCRLFYGETEYGAYLPVKEVARALFHGIGFEGWVSLELFNRRMSEEGPEVPEELAMRGAISWAKLVQDLRIPVEGPLVTMPRVSASL

SEQ ID NO: 2 - protein, 351 aa, Unknown
  Description of Unknown: Gibberella zeae strain PH-1 sequence
  3-dehydroshikimate dehydratase
MAHKPSICTMSLGRCFAGHSLPHKLDMAAKYGFQGIEVFYEDLVDLSKSLPGGATHANQVIAARTIHDLCQDRSLDIICLQPFMHFGGLVDRDAQEKQFDELRHWFDLVHALDTDLILFPSSFLPAEQTTDDINVLTSDFTRAAEMGLQQQPVVRFAFEALCWGTRLSLWEESWEMVQRVDRSNFGMCLDTYNILGRIYADPATESGKTSDCDQVTQDSIKKLLTEVDVSRVFLLQVADGEKLNSPLNESHPFYNAEQPSRMSWSRNARLFYGESSYGAYLPSKQLLRAIVQGLGFEGWLSFEVFNRRFLDTDKIVPEETAKRAAESFEKMKIDIGLRTSDQVQPRLQANL

SEQ ID NO: 3 - protein, 370 aa, Saccharomyces cerevisiae
  DAHP synthase
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 4 - protein, 365 aa, Corynebacterium glutamicum
  3-dehydroquinate synthase
MSAVQIFNTVHVNGSSPYDVHIGSGLNELIVQRAAESGAEQVAILHQPSMDDIASELDAALVAAGLKVLHLNVPDAENGKSLEVAGQCWDELGGAAFGRRDIVIGLGGGAATDLAGFVAAAWMRGVRVIQVPTTLLAMVDAAVGGKTGINTAAGKNLVGAFHEPDAVFIDTDRLATLPDAEIIAGSAEIIKTGFIADPEILRLYETDPAACLKKEVEGSHLPELIWRSVTVKGSVVGQDLKESSLREILNYGHTFAHAVELRENFRWRHGNAVAVGMMFIANLSHKLGLIDAPLLERHRSILAAIGLPTSYEGGAFDELYDGMTRDKKNRDGNIRFVALTAVGEVTRIEGPSKQDLQSAYEAISH

SEQ ID NO: 5 - protein, 333 aa, Saccharomyces cerevisiae
  Transaldolase
MSEPSEKKQKVATSSLEQLKKAGTHVVADSGDFEAISKYEPQDSTTNPSLILAASKLEKYARFIDAAVEYGRKHGKTDHEKIENAMDKILVEFGTQILKVVPGRVSTEVDARLSFDKKATVKKALHIIKLYKDAGVPKERVLIKIASTWEGIQAARELEVKHGIHCNMTLLFSFTQAVACAEANVTLISPFVGRIMDFYKALSGKDYTAETDPGVLSVKKIYSYYKRHGYATEVMAASFRNLDELKALAGIDNMTLPLNLLEQLYESTDPIENKLNSESAKEEGVEKVSFINDEPHFRYVLNEDQMATEKLSDGIRKFSADIEALYKLVEEKM

SEQ ID NO: 6 - protein, 1588 aa, Saccharomyces cerevisiae
  3-dehydroquinate synthase,3-phosphoshikimate 1-carboxyvinyltransferase,3-phosphoshikimate 1-carboxyvinyltransferase,Shikimate kinase (SK),Shikimate 5-dehydrogenase,3-dehydroquinate dehydratase (3-dehydroquinase)
MVQLAKVPILGNDIIHVGYNIHDHLVETIIKHCPSSTYVICNDTNLSKVPYYQQLVLEFKASLPEGSRLLTYVVKPGETSKSRETKAQLEDYLLVEGCTRDTVMVAIGGGVIGDMIGFVASTFMRGVRVVQVPTSLLAMVDSSIGGKTAIDTPLGKNFIGAFWQPKFVLVDIKWLETLAKREFINGMAEVIKTACIWNADEFTRLESNASLFLNVVNGAKNVKVTNQLTNEIDEISNTDIEAMLDHTYKLVLESIKVKAEVVSSDERESSLRNLLNFGHSIGHAYEAILTPQALHGECVSIGMVKEAELSRYFGILSPTQVARLSKILVAYGLPVSPDEKWFKELTLHKKTPLDILLKKMSIDKKNEGSKKKVVILESIGKCYGDSAQFVSDEDLRFILTDETLVYPFKDIPADQQKVVIPPGSKSISNRALILAALGEGQCKIKNLLHSDDTKHMLTAVHELKGATISWEDNGETVVVEGHGGSTLSACADPLYLGNAGTASRFLTSLAALVNSTSSQKYIVLTGNARMQQRPIAPLVDSLRANGTKIEYLNNEGSLPIKVYTDSVFKGGRIELAATVSSQYVSSILMCAPYAEEPVTLALVGGKPISKLYVDMTIKMMEKFGINVETSTTEPYTYYIPKGHYINPSEYVIESDASSATYPLAFAAMTGTTVTVPNIGFESLQGDARFARDVLKPMGCKITQTATSTTVSGPPVGTLKPLKHVDMEPMTDAFLTACVVAAISHDSDPNSANTTTIEGIANQRVKECNRILAMATELAKFGVKTTELPDGIQVHGLNSIKDLKVPSDSSGPVGVCTYDDHRVAMSFSLLAGMVNSQNERDEVANPVRILERHCTGKTWPGWWDVLHSELGAKLDGAEPLECTSKKNSKKSVVIIGMRAAGKTTISKWCASALGYKLVDLDELFEQQHNNQSVKQFVVENGWEKFREEETRIFKEVIQNYGDDGYVFSTGGGIVESAESRKALKDFASSGGYVLHLHRDIEETIVFLQSDPSRPAYVEEIREVWNRREGWYKECSNFSFFAPHCSAEAEFQALRRSFSKYIATITGVREIEIPSGRSAFVCLTFDDLTEQTENLTPICYGCEAVEVRVDHLANYSADFVSKQLSILRKATDSIPIIFTVRTMKQGGNFPDEEFKTLRELYDIALKNGVEFLDLELTLPTDIQYEVINKRGNTKIIGSHHDFQGLYSWDDAEWENRFNQALTLDVDVVKFVGTAVNFEDNLRLEHFRDTHKNKPLIAVNMTSKGSISRVLNNVLTPVTSDLLPNSAAPGQLTVAQINKMYTSMGGIEPKELFVVGKPIGHSRSPILHNTGYEILGLPHKFDKFETESAQLVKEKLLDGNKNFGGAAVTIPLKLDIMQYMDELTDAAKVIGAVNTVIPLGNKKFKGDNTDWLGIRNALINNGVPEYVGHTAGLVIGAGGTSRAALYALHSLGCKKIFIINRTTSKLKPLIESLPSEFNIIGIESTKSIEEIKEHVGVAVSCVPADKPLDDELLSKLERFLVKGAHAAFVPTLLEAAYKPSVTPVMTISQDKYQWHVVPGSQMLVHQGVAQFEKWTGFKGPFKAIFDAVTKE

SEQ ID NO: 7 - protein, 437 aa, Saccharomyces cerevisiae
  Enolase
MAVSKVYARSVYDSRGNPTVEVELTTEKGVFRSIVPSGASTGVHEALEMRDGDKSKWMGKGVLHAVKNVNDVIAPAFVKANIDVKDQKAVDDFLISLDGTANKSKLGANAILGVSLAASRAAAAEKNVPLYKHLADLSKSKTSPYVLPVPFLNVLNGGSHAGGALALQEFMIAPTGAKTFAEALRIGSEVYHNLKSLTKKRYGASAGNVGDEGGVAPNIQTAEEALDLIVDAIKAAGHDGKIKIGLDCASSEFFKDGKYDLDFKNPNSDKSKWLTGPQLADLYHSLMKRYPIVSIEDPFAEDDWEAWSHFFKTAGIQIVADDLTVTNPKRIATAIEKKAADALLLKVNQIGTLSESIKAAQDSFAAGWGVMVSHRSGETEDTFIADLVVGLRTGQIKTGAPARSERLAKLNQLLRIEEELGDNAVFAGENFHHGDKL

SEQ ID NO: 8 - protein, 360 aa, Corynebacterium glutamicum
  Transaldolase
MSHIDDLAQLGTSTWLDDLSRERITSGNLSQVIEEKSVVGVTTNPAIFAAAMSKGDSYDAQIAELKAAGASVDQAVYAMSIDDVRNACDLFTGIFESSNGYDGRVSIEVDPRISADRDATLAQAKELWAKVDRPNVMIKIPATPGSLPAITDALAEGISVNVTLIFSVARYREVIAAFIEGIKQAAANGHDVSKIHSVASFFVSRVDVEIDKRLEAIGSDEALALRGKAGVANAQRAYAVYKELFDAAELPEGANTQRPLWASTGVKNPAYAATLYVSELAGPNTVNTMPEGTIDAVLEQGNLHGDTLSNSAAEADAVFSQLEALGVDLADVFQVLETEGVDKFVASWSELLESMEARLK

SEQ ID NO: 9 - protein, 145 aa, Corynebacterium glutamicum
  3-dehydroquinate dehydratase (3-dehydroquinase)
MPGKILLLNGPNLNMLGKREPDIYGHDTLEDVVALATAEAAKHGLEVEALQSNHEGELIDALHNARGTHIGCVINPGGLTHTSVALLDAVKASELPTVEVHISNPHAREEFRHHSYISLAAVSVIAGAGIQGYRFAVDILANLKK

SEQ ID NO: 10 - DNA, 1077 bp, Unknown
  Description of Unknown: Neurospora crassa ATCC 24698 sequence
  3-dehydroshikimate dehydratase
atgccttccaagctggcaatctcctccatgtccttgggtcgttgcttcgcaggccactccttggattccaagttggatgcagcacagcgttacggctacctgggcatcgaactgttctacgaagatctggtggatgtggcagaacacctgtccaacgaacgtccatctcctgaaggcccattcgtggaagcacagatcgcagcagcacgtcacatcctgcaaatgtgccaggcacgtggcttggaagtggtgtgcctgcagccattcatgcactacgatggcctgaacgatcgtgcagaacacgaacgtcgtctggaaaagctggcactgtggatcgaactggcacacgaactgcacaccgatatcatccagattccagcaaacttcctgccagcaaaccaggtgtccgataacctggatctgatcgtgtccgatttgtgcaaggtggcagatattggcgcacaggcactgccaccaatccgtttcgcatacgaatccctgtgctggtctacccgtgtggatctgtgggaacgttgctgggatatcgtgcagcgtgtggaccgtccaaacttcggcatctgcctggataccttcaacatcctgggtcgtatctacgcagatccaacctctccatccggtcgtaccccaaacgcaaaagaagcagtgcgtaagtccattgcaaacttggtgtcccgtgtcgacgtgtccaaggtgttctacgtgcaggtcgtggatgcagaacgtctgtccaagccattgctgccaggtcacccatactacaacccagaacagccagcacgtatgtcttggtcccgtaactgccgtttgttctacggcgaaaccgaatacggtgcatacctgccagtgaaagaagtggcacgcgcactgttccacggcatcggcttcgaaggctgggtgtccctggaactgttcaaccgtcgtatgtccgaagaaggcccagaagtcccagaggaactggcaatgcgtggcgctatctcctgggcaaagctggtgcaggatttgcgtatcccagtggaaggcccactggtgaccatgccacgtgtgtccgcatccttg

SEQ ID NO: 11 - DNA, 1053 bp, Unknown
  Description of Unknown: Gibberella zeae strain PH-1 sequence
  3-dehydroshikimate dehydratase
atggcacacaagccatccatctgcaccatgtccttgggtcgttgcttcgcaggccactccttgccacacaagctggatatggcagcaaagtacggcttccagggcatcgaagtgttctacgaagatctggtggatctgtccaagtccttgccaggcggtgcaacccacgcaaaccaggtgatcgcagcacgtaccatccacgatctgtgccaggatcgttccctggatatcatctgcctgcagccattcatgcacttcggtggcttggtggatcgtgatgcacaagaaaagcagttcgatgaattgcgtcactggttcgatctggtgcacgcactggataccgatctgatcttgttcccgtcctccttcctgccagcagaacagaccaccgatgatatcaacgtgctgacctccgatttcacccgtgcagcagaaatgggcctgcagcagcagccagtggtgcgtttcgcattcgaagcactgtgctggggcacccgtctgtccctgtgggaagaatcctgggaaatggtgcagcgtgtggaccgttccaacttcggcatgtgcctggatacctacaacatcctgggtcgtatctacgcagatccagcaaccgaatccggcaagacctccgactgcgatcaggtgacccaggactccatcaagaaactgctgaccgaagtggatgtgtcccgtgtgttcctgttgcaggtcgcagatggcgaaaagctgaactccccactgaacgaatctcacccattctacaacgcagaacagccatctcgtatgtcttggtcccgtaacgcacgtctgttctacggcgaatcctcctacggtgcatacctgccatccaagcagctgttgcgtgcaatcgtgcaaggcctgggcttcgaaggctggctgtccttcgaggtgttcaaccgtcgtttcctggacaccgataagatcgtgcctgaagaaaccgcaaagcgtgcagccgaatccttcgaaaagatgaagatcgatatcggcctgcgtacctccgaccaggtgcagccacgtctgcaggcaaacctg

SEQ ID NO: 12 - DNA, 1110 bp, Saccharomyces cerevisiae
  DAHP synthase
atgtccgaatctccaatgttcgctgccaacggcatgccgaaggtgaaccagggagccgaagaagacgtgcgcatcctcggttatgatccgctcgccagcccggcactcttgcaagtgcagattcccgctacgccaacctcactcgagaccgcgaagcgcggtcgccgcgaagcaatcgacatcattactggtaaggacgatcgcgtgctcgtgatcgttggcccgtgctcgattcacgacttggaagcggctcaagagtacgcgcttcgcctcaagaaattgagcgatgaactgaaaggtgatttgtccatcattatgcgcgcgtaccttgaaaagccacgtacgactgttggttggaagggtctgatcaacgatcctgatgtcaacaacacctttaatattaataagggcctccagtccgcccgccaacttttcgtgaacctgaccaacattggccttccaattggctcagagatgcttgacacgatctctccacaatacctcgcagatttggtcagcttcggcgcaatcggcgcgcgcaccaccgagtcccagctccaccgcgaactggcgagcggactgagctttcccgtcggcttcaagaatggcacagatggcaccttgaacgtcgcggtggacgcgtgtcaagccgccgcgcattcacaccacttcatgggagtgaccttgcacggtgtagcggccatcactaccacgaagggtaacgagcactgcttcgtgatcttgcgtggaggtaagaagggaactaattacgacgccaagtctgtggcggaggctaaggctcagttgccagcgggatccaacggtttgatgatcgattatagccacggcaactccaacaaagactttcgtaatcaacctaaggtgaacgatgtagtctgcgaacaaatcgcgaacggcgaaaacgcgatcaccggtgtgatgatcgaatctaatattaacgaaggtaaccagggtattcccgcggagggcaaggcaggtctgaaatacggagtcagcattacggatgcttgcatcggatgggagaccaccgaagatgttctccgaaagttggcggcagcggtgcgccagcgccgcgaagtgaataagaaa

SEQ ID NO: 13 - DNA, 1095 bp, Corynebacterium glutamicum
  3-dehydroquinate synthase
atgagcgcagttcagatctttaacactgtgcacgtgaacggctcttccccctacgatgtgcacatcggttccggtttgaacgaactcattgtccaacgcgccgctgagagcggcgccgaacaggttgcgatcctgcatcagccatctatggatgatatcgcgtctgagctggatgcagcccttgtcgcagctggcttgaaggtgctgcacttgaacgttcctgatgcggagaacggtaagtcgctggaggtggcgggccaatgctgggacgaactgggtggtgcagctttcggacgtcgtgacatcgtgattggtctgggaggcggtgccgcgacagatcttgcaggctttgtggccgcggcgtggatgcgcggcgttcgtgttatccaggttccgacaacacttcttgccatggtggacgccgcagtgggcggtaaaaccggaatcaacactgcggcaggcaagaacctggttggcgcgttccacgaaccagatgcagtttttattgatacagaccgcctcgctacgctgcctgatgcagaaattattgcgggttccgcagagattattaagactggctttatcgccgatccagaaattctgcgactctacgaaaccgaccctgccgcatgccttaagaaggaggtggagggctcccatctgcctgaacttatctggcgatctgtaaccgtcaagggtagcgtcgtgggtcaggatcttaaggagtcgtccctccgagaaattttgaactatggccacaccttcgcccatgcggtcgaacttcgagaaaattttcgctggcgccacggcaacgctgtcgccgttggcatgatgttcattgccaatctgtcgcacaagcttggcctcatcgatgccccactgctggagcgccatcgctcaatcttggccgcgatcggactgccaactagctacgaaggcggtgcatttgacgaactctatgacggtatgacccgtgataaaaaaaaccgagacggtaatatccgtttcgtggcccttaccgcagtaggcgaagtcacccgtatcgaaggcccgtccaaacaggacctgcagagcgcgtatgaagcgatctcccac

SEQ ID NO: 14 - DNA, 999 bp, Saccharomyces cerevisiae
  Transaldolase
atgtctgaaccctcggaaaagaagcaaaaggtggcaacttcttccttggagcagctcaagaaagctggcacccacgttgtcgctgatagcggtgatttcgaggccatttccaagtacgaacctcaggattccacgaccaacccatctcttatcttggcggcatcgaagcttgagaaatatgcacgatttatcgatgctgctgtcgagtacggccgtaaacacggaaaaaccgatcacgagaaaattgagaatgccatggataaaatcctggttgagtttggcacccagattctcaaggtggtacccggccgcgtctccaccgaagtagacgctcgcctgtcttttgataagaaagctacagttaagaaggctttgcatatcattaagctttataaggatgctggagttcctaaggagcgcgttcttatcaagatcgcaagcacgtgggagggtattcaggcagcccgtgaactcgaagtcaaacatggtattcactgcaacatgacactcttgttctctttcacgcaggcagttgcatgcgcagaagccaacgtgaccctcatttcgccgttcgtgggtcgaattatggacttctacaaggcactctcaggtaaggactataccgccgaaacagatccaggcgtgctgtccgtgaagaaaatttacagctactataagcgccacggctacgcgaccgaagtcatggcggcatcgttccgcaaccttgacgagttgaaggcattggcaggcattgacaatatgacccttcctcttaacctcttggaacaactctacgagtccaccgatccgattgaaaataagctcaattccgagtccgcgaaggaggagggagtcgaaaaagtgtccttcattaatgatgaaccacatttccgttacgtgcttaacgaagaccaaatggccaccgagaagctgagcgatggaattcgtaagttttccgccgatattgaagcactgtacaaactcgtcgaagagaaaatg

SEQ ID NO: 15 - DNA, 4764 bp, Saccharomyces cerevisiae
  3-dehydroquinate synthase,3-phosphoshikimate 1-carboxyvinyltransferase,3-phosphoshikimate 1-carboxyvinyltransferase,Shikimate kinase (SK),Shikimate 5-dehydrogenase,3-dehydroquinate dehydratase (3-dehydroquinase)
atggtccaacttgctaaagtgccgatcttgggtaacgatattattcatgtgggctacaatatccatgatcacctcgtggaaaccatcatcaagcactgtccatctagcacgtacgtcatttgcaatgacactaacttgtcgaaggtgccgtattatcaacagcttgtgcttgagtttaaggcgtcgctccccgaaggtagccgtctcctcacctacgtggtcaaaccgggagagacgtcgaaaagccgtgaaaccaaggcccagctcgaggactatctcttggtcgagggatgcacccgagataccgtcatggttgcgatcggaggaggagtgattggtgatatgatcggtttcgtggcatcaacattcatgcgtggcgtgcgcgtggtccaagtgccaacctcgctcctggcaatggtggacagctccatcggaggcaaaaccgctattgataccccattgggtaagaactttatcggtgcgttctggcagccaaaattcgtattggtagacattaagtggttggagaccttggctaagcgtgaatttatcaacggcatggccgaagttatcaaaactgcgtgcatttggaatgcggatgaatttacacgcctggaatcaaacgcctcactctttcttaatgtcgtgaacggcgctaaaaacgtcaaagtcaccaaccaacttactaatgaaatcgacgaaatcagcaacacggacatcgaagcgatgctcgatcatacctacaagttggtgttggaatcaattaaagtcaaagcagaagttgtctcctctgatgaacgcgagtcctctctgcgcaacctcttgaacttcggtcactctatcggccacgcttacgaggctatcttgaccccgcaagcactgcacggcgaatgtgtctctattggcatggttaaggaggcagagctttcccgttatttcggcattctgtccccaacccaggtggcccgcctgagcaagatcctcgttgcgtacggcctgccagtgtctcccgatgaaaaatggtttaaggagctgactttgcacaaaaagaccccactggacatcctgctgaagaaaatgtccatcgataagaagaacgaaggtagcaagaagaaagtcgttatcttggaatccatcggcaagtgctacggcgactccgcacaatttgtgtcggacgaggacctgcgctttatcctgaccgacgaaacgttggtctacccatttaaggatatcccagcagatcaacaaaaggtcgtgattcctccaggaagcaaatccatttccaaccgcgcattgattctggcggccctcggcgaaggacagtgcaaaattaaaaatctgctgcattccgacgacaccaagcacatgctcactgcggtgcacgagctcaaaggtgcaaccatcagctgggaagataacggagagaccgtcgtggtcgagggccacggcggcagcacgctgtctgcttgcgccgatccgctgtatcttggcaatgcaggcactgcttcgcgcttcctgacctcccttgccgcactcgtaaacagcacctcctcccagaaatacatcgtgttgaccggtaatgcacgcatgcaacagcgaccaatcgctccgcttgtggactccctgcgtgccaacggcaccaaaatcgaatacctcaacaatgaaggctccctgccaattaaggtttacaccgattccgtattcaagggtggtcgtattgagctcgccgccaccgtctcgagccagtacgtctctagcattcttatgtgcgcgccatacgccgaagaaccagttacgctggcgctcgtgggcggtaaaccaatctcgaagctgtacgttgatatgactatcaagatgatggaaaaattcggtattaacgtcgaaacgtccacaactgaaccatatacttactatatcccaaaaggccactacattaatccctccgagtacgttatcgagtcagacgcaagctccgcaacgtacccactggcatttgctgcgatgaccggtaccactgtgactgtcccgaacattggcttcgaaagcttgcaaggcgacgctcgctttgcacgtgatgtccttaaaccaatgggctgtaaaattacccagactgccacctcaaccaccgtatccggcccccctgtaggcaccttgaagcccctcaaacatgtggatatggagccaatgactgacgctttcctgaccgcatgcgtagtcgctgccatttcccacgattcagatccaaattctgcgaacaccacgacaatcgagggtattgctaaccaacgtgttaaggaatgcaatcgaattctcgccatggcaacggaattggcaaaattcggtgtgaaaactacggaattgccagatggaattcaagtgcacggactcaactcgatcaaggatcttaaggtcccatcggactcctccggcccagtgggtgtgtgtacctatgatgatcatcgtgtggccatgtccttttcactgctcgcaggaatggttaactcccaaaacgaacgtgatgaggttgccaaccctgttcgcatcctcgagcgccactgcaccggtaagacttggccaggttggtgggacgtactgcactctgagcttggcgctaaactggatggcgctgaacccctcgagtgcacttcaaaaaaaaactccaaaaagtccgtcgtcattattggcatgcgagcggccggtaagacaaccatttcaaagtggtgtgcctccgcgttgggctacaaacttgtagaccttgacgagctttttgagcaacaacacaataatcagagcgttaaacagttcgtcgttgaaaacggttgggaaaagtttcgcgaggaagaaacccgaatcttcaaggaggttatccagaactacggcgacgacggttacgtgttcagcaccggtggcggaattgtggagtcggctgagtcgcgcaaggccctgaaggacttcgcttctagcggcggatatgtgcttcacctgcatcgcgatattgaggagaccattgtgttcctccagtccgaccctagccgccctgcatacgttgaggagattcgagaagtgtggaaccgccgagaaggttggtacaaggaatgttcaaatttttccttcttcgctcctcactgctctgctgaagccgaattccaagccctgcgccgctcgttttcgaaatatatcgctacaattaccggagtgcgagaaatcgagattccgtcgggacgtagcgcattcgtctgtctgacattcgatgatctgactgaacaaaccgagaacctgacgcctatttgttacggttgtgaagcagtggaagttcgcgttgatcacctcgccaactactcggcagacttcgtatcaaagcaactgtcaatcttgcgtaaagctacagatagcatccctatcattttcaccgttcgcacaatgaaacagggcggtaatttccctgatgaagagttcaaaaccttgcgcgagctctatgacatcgcgttgaagaacggagtagaatttttggatctcgagctgacgctgccaaccgatattcaatacgaggttattaacaaacgcggcaataccaagatcatcggctctcaccacgactttcagggcctgtactcttgggatgacgcggaatgggagaaccgcttcaaccaggcgctcaccctggacgtcgatgtcgtgaagttcgttggtacggcagtgaactttgaggataatttgcgcttggaacatttccgtgatacccacaagaacaagccccttatcgcggttaacatgacctcaaaaggctctatctcacgtgtgctgaacaacgtcctgactcctgttacctccgatctgctgccaaatagcgcggcgcctggacagctcactgttgcgcaaattaacaaaatgtacacttccatgggtggcatcgaacctaaggaactcttcgttgttggaaagcctatcggtcactcacgctcccctatccttcacaacaccggttacgagattctgggattgccccacaagttcgacaaatttgaaacggaatccgcacagttggtgaaagaaaagctgctggacggtaacaagaatttcggcggagcggctgtcaccatccctcttaagctggacatcatgcaatacatggatgagctcaccgacgcagcaaaagtgatcggtgctgtgaatactgtcatccctctgggtaacaagaaattcaaaggcgataacacggattggctgggcatccgcaatgcactgatcaataacggagtgccagagtacgtcggccacactgcgggcttggttattggcgcgggcggtacttcccgcgcagcgctgtacgctttgcactccctcggttgtaagaagatcttcattattaaccgcaccacgtctaagctcaagccgcttattgaatccttgccttccgaattcaacatcatcggaatcgagtccactaagagcattgaagaaatcaaggaacacgttggcgttgctgtctcctgtgtgccagcagataagccgttggatgacgagctcctcagcaagctggagcgcttcttggtgaagggagcacatgcggccttcgtcccaaccttgttggaggcagcgtacaagccgtccgtaactccagtcatgaccattagccaggataagtatcagtggcacgttgttccgggctcccaaatgctggtgcaccaaggtgtagcacagttcgagaagtggaccggttttaaaggtccgttcaaagctattttcgacgcagtgacgaaggag

SEQ ID NO: 16 - DNA, 1311 bp, Saccharomyces cerevisiae
  Enolase
atggcggtgtctaaggtgtacgctcgttccgtctatgattctcgaggtaacccgactgtggaggttgaactgaccacagagaagggcgtcttccgatccatcgtgccgagcggagcatccaccggtgtgcacgaagcccttgagatgcgtgatggagataaatctaagtggatgggcaagggcgtacttcacgccgttaaaaacgttaacgacgtaattgcccctgcttttgttaaagcaaacatcgacgtgaaggatcagaaggccgtcgatgattttcttatctcgctcgacggcaccgctaacaaatccaagctcggcgcaaacgcaatcctgggtgtctcattggcagcctcacgcgcagcggcagctgagaaaaacgtacccctctataaacacctggcggaccttagcaaatccaaaacctcaccatacgtcctgcccgtcccattcctcaatgtattgaacggtggatcccacgctggaggtgccctcgctcttcaggaatttatgattgcccctaccggcgcaaagacgtttgcggaggcactccgcatcggctccgaagtctaccataacctcaagtctctcaccaaaaagcgctatggtgcgtccgccggcaatgtcggcgacgaaggtggcgttgctccaaacattcagactgccgaagaggcacttgacttgatcgttgatgccatcaaagctgctggccacgacggcaagatcaagattggtctggattgcgccagctccgaattctttaaagacggtaagtatgatctggatttcaaaaacccgaattccgataagagcaagtggctcaccggcccgcaattggcagacttgtatcactcgctgatgaaacgctaccccatcgtcagcatcgaggatccgttcgctgaagacgactgggaagcgtggagccactttttcaagaccgcaggcattcagatcgtagcagacgatcttaccgtaactaatcccaagcgtatcgctactgctatcgagaagaaggcagctgatgcgctgctgctgaaagttaaccagattggcaccctgtcggagtcgatcaaggctgcccaggacagctttgcagctggttggggtgttatggtaagccaccgctcaggtgaaaccgaagataccttcattgccgacctggtggtgggcttgcgaactggacagatcaaaactggcgcacctgcacgcagcgaacgactcgcaaagttgaatcaactgctgcgtattgaagaagaactgggcgacaacgcagtctttgcgggcgaaaacttccaccacggagataaactc

SEQ ID NO: 17 - DNA, 1080 bp, Corynebacterium glutamicum
  Transaldolase
atgtctcatatcgacgacctggcgcaactgggcacttcgacttggctggatgatctctcgcgtgagcgtatcacctccggtaacctttcgcaggttattgaagagaagtcggtcgttggcgtcaccacaaacccagcaatcttcgcagctgctatgtcgaaaggagactcttacgacgcccaaatcgcagagctgaaagcagcaggtgcgtccgtggaccaagcagtttacgcaatgtctatcgacgatgttcgtaacgcatgtgatcttttcaccggcatttttgagtcttccaacggttacgatggtcgcgtctccattgaggttgaccctcgaatctctgctgatcgagacgccacgctcgctcaggctaaggagctctgggcgaaagttgatcgtccaaacgtgatgatcaaaattcctgctactcctggatccttgcccgctatcaccgatgctcttgcagaaggtatttcagttaacgtgaccttgatcttcagcgttgcgcgttatcgcgaagtcatcgccgcgttcatcgaaggtattaagcaagcagccgctaacggccacgacgtttctaagatccactcagtggcttccttcttcgtctcgcgcgtggacgttgagatcgacaagcgcctcgaggccatcggctctgatgaagccctggctttgcgcggcaaggccggcgtcgccaatgcacagcgtgcgtatgcagtctacaaggaacttttcgacgccgcagagcttccggaaggcgcaaacacccagcgtccactctgggcatcaactggcgtcaaaaaccccgcatacgcggcaaccctttacgtgtccgagcttgcaggcccaaacaccgttaacaccatgcccgaaggcaccattgatgcggtgctcgagcagggtaacctgcacggcgatacacttagcaattcagctgctgaagccgatgcggtgttctcacagctcgaagcactcggcgtcgatttggcagatgtattccaggtgcttgagaccgagggcgtggataagttcgtggccagctggagcgaattgttggaaagcatggaagcacgactcaaa

SEQ ID NO: 18 - DNA, 435 bp, Corynebacterium glutamicum
  3-dehydroquinate dehydratase (3-dehydroquinase)
atgccaggcaagatccttctgttgaatggcccaaacttgaatatgcttggcaagcgtgagcccgatatttatggccatgacactctcgaggatgttgtcgctctcgctaccgcagaggcagcgaagcacggcctggaagttgaagcactgcagtctaaccatgaaggagaacttattgacgcgttgcacaatgcacgcggtacgcatattggctgcgttatcaacccaggcggcctcactcacacgtccgtggcattgcttgacgctgtcaaagcgtccgaactccctaccgtcgaggtgcacatttccaacccacacgcccgcgaggaattccgccaccactcttacatctcccttgctgcagtctctgtgatcgcaggtgccggaattcagggataccgctttgcggtcgacatccttgcgaatctcaaaaaa

(19) United States
(12) Patent Application Publication
(10) Pub. No.: US 2023/0175023 A1
(43) Pub. Date: Jun. 8, 2023

(54) ENGINEERED BIOSYNTHETIC PATHWAYS FOR PRODUCTION OF 3,4-DIHYDROXYBENZOIC ACID BY FERMENTATION

(71) Applicant: Zymergen Inc., Emeryville, CA (US)

(72) Inventors: Cara Ann Tracewell, Walnut Creek, CA (US); Alexander Glennon Shearer, San Francisco, CA (US); Anupam Chowdhury, Emeryville, CA (US)

(21) Appl. No.: 17/917,221

(22) PCT Filed: Feb. 18, 2021
(86) PCT No.: PCT/US2021/018609

§ 371 Date: Oct. 5, 2022

(51) Int. Cl. C12P 7/42; C12N 9/88; C12N 9/10

(52) U.S. Cl. CPC C12P 7/42; C12N 9/88; C12N 9/1022; C12N 9/1085; C12Y 202/01002; C12Y 205/01054; C12Y 402/0101; C12Y 402/01011; C12Y 402/01118; C12Y 402/03004

(57) ABSTRACT

The present disclosure describes the engineering of microbial cells for fermentative production of 3,4-dihydroxybenzoic acid and provides novel engineered microbial cells and cultures, as well as related 3,4-dihydroxybenzoic acid production methods.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Feb. 18, 2021, is named ZMGNP008WO_SL.txt and is 57,044 bytes in size.

### FIELD OF THE DISCLOSURE

The present disclosure relates generally to the area of engineering microbes for production of 3,4-dihydroxybenzoic acid by fermentation.

### BACKGROUND

3,4-dihydroxybenzoic acid is known to exist in nature and is found in some plants, such as acai fruit, the extract of which contains 630 mg/kg [1].

3,4-dihydroxybenzoic acid can derived from the shikimate pathway metabolite, 3-dehydroshikimate. This metabolite can be converted to 3,4-dihydroxybenzoic acid by a 3-dehydroshikimate.

### SUMMARY

The disclosure provides engineered microbial cells, cultures of the microbial cells, and methods for producing 3,4-dihydroxybenzoic acid, including the following:

Various embodiments contemplated herein may include, but need not be limited to, one or more of the following:

Embodiment 1: An engineered microbial cell that expresses a non-native 3-dehydroshikimate dehydratase, wherein the engineered microbial cell produces 3,4-dihydroxybenzoic acid.

Embodiment 2: The engineered microbial cell of embodiment 1, wherein the engineered microbial cell includes increased activity of one or more upstream 3,4-dihydroxybenzoic acid pathway enzyme(s), said increased activity being increased relative to a control cell.

Embodiment 3: The engineered microbial cell of embodiment 2, wherein the one or more upstream 3,4-dihydroxybenzoic acid pathway enzyme(s) are selected from the group consisting of an enolase, a transketolase, a transaldolase, phospho-2-dehydro-3-deoxyheptonate aldolase, a 3-deoxy-D-arabino-heptulosonate-7-phosphate (DAHP) synthase, a 3-dehydroquinate synthase, and a 3-dehydroquinate dehydratase.

Embodiment 4: The engineered microbial cell of embodiment 3, wherein the one or more upstream 3,4-dihydroxybenzoic acid pathway enzyme(s) are selected from the group consisting of an enolase, a transaldolase, a 3-dehydroquinate synthase, and a 3-dehydroquinate dehydratase.

Embodiment 5: The engineered microbial cell of any one of embodiments 1-4, wherein the engineered microbial cell includes reduced activity of one or more enzyme(s) that consume one or more 3,4-dihydroxybenzoic acid pathway precursors, said reduced activity being reduced relative to a control cell.

Embodiment 6: The engineered microbial cell of embodiment 5, wherein the one or more enzyme(s) that consume one or more 3,4-dihydroxybenzoic acid pathway precursors comprise shikimate:NADP+ 3-oxidoreductase.

Embodiment 7: The engineered microbial cell of embodiment 5 or embodiment 6, wherein the reduced activity is achieved by replacing a native promoter of a gene for said one or more enzymes with a less active promoter.

Embodiment 8: The engineered microbial cell of any one of embodiments 1-7, wherein the engineered microbial cell additionally expresses a feedback-deregulated DAHP synthase.

Embodiment 9: An engineered microbial cell, wherein the engineered microbial cell includes means for expressing a non-native 3-dehydroshikimate dehydratase, wherein the engineered microbial cell produces 3,4-dihydroxybenzoic acid.

Embodiment 10: The engineered microbial cell of embodiment 9, wherein the engineered microbial cell includes means for increasing the activity of one or more upstream 3,4-dihydroxybenzoic acid pathway enzyme(s), said increased activity being increased relative to a control cell.

Embodiment 11: The engineered microbial cell of embodiment 10, wherein the one or more upstream 3,4-dihydroxybenzoic acid pathway enzyme(s) are selected from the group consisting of an enolase, a transketolase, a transaldolase, phospho-2-dehydro-3-deoxyheptonate aldolase, a 3-deoxy-D-arabino-heptulosonate-7-phosphate (DAHP) synthase, a 3-dehydroquinate synthase, and a 3-dehydroquinate dehydratase.

Embodiment 12: The engineered microbial cell of embodiment 11, wherein the one or more upstream 3,4-dihydroxybenzoic acid pathway enzyme(s) are selected from the group consisting of an enolase, a transaldolase, a 3-dehydroquinate synthase, and a 3-dehydroquinate dehydratase.

Embodiment 13: The engineered microbial cell of any one of embodiments 9-12, wherein the engineered microbial cell includes means for reducing the activity of one or more enzyme(s) that consume one or more 3,4-dihydroxybenzoic acid pathway precursors, said reduced activity being reduced relative to a control cell.

Embodiment 14: The engineered microbial cell of embodiment 13, wherein the one or more enzyme(s) that consume one or more 3,4-dihydroxybenzoic acid pathway precursors comprise shikimate:NADP+ 3-oxidoreductase.

Embodiment 15: The engineered microbial cell of embodiment 13 or embodiment 14, wherein the reduced activity is achieved by means for replacing a native promoter of a gene for said one or more enzymes with a less active promoter.

Embodiment 16: The engineered microbial cell of any one of embodiments 9-15, wherein the engineered microbial cell additionally includes means for expressing a feedback-deregulated DAHP synthase.

Embodiment 17: The engineered microbial cell of any one of embodiments 1-16, wherein the engineered microbial cell includes a fungal cell.

Embodiment 18: The engineered microbial cell of embodiment 17, wherein the engineered microbial cell includes a yeast cell.

Embodiment 19: The engineered microbial cell of embodiment 18, wherein the yeast cell is a cell of the genus Saccharomyces.

Embodiment 20: The engineered microbial cell of embodiment 19, wherein the yeast cell is a cell of the species cerevisiae.

Embodiment 21: The engineered microbial cell of any one of embodiments 1-20, wherein the non-native 3-dehydroshikimate dehydratase includes a 3-dehydroshikimate dehydratase having at least 70% amino acid sequence identity with a 3-dehydroshikimate dehydratase from an organism selected from the group consisting of Neurospora crassa, Corynebacterium glutamicum, Bacillus anthracis, and Gibberella zeae.

Embodiment 22: The engineered microbial cell of embodiment 21, wherein the non-native 3-dehydroshikimate dehydratase includes a 3-dehydroshikimate dehydratase having at least 70% amino acid sequence identity with a 3-dehydroshikimate dehydratase from Neurospora crassa.

Embodiment 23: The engineered microbial cell of embodiment 21, wherein the non-native 3-dehydroshikimate dehydratase includes a 3-dehydroshikimate dehydratase having at least 70% amino acid sequence identity with a 3-dehydroshikimate dehydratase from Corynebacterium glutamicum.

Embodiment 24: The engineered microbial cell of any one of embodiments 4 or 12-23, wherein the increased activity of the enolase is achieved by heterologously expressing an enolase.

Embodiment 25: The engineered microbial cell of embodiment 24, wherein the heterologous enolase includes an enolase from Saccharomyces cerevisiae.

Embodiment 26: The engineered microbial cell of any one of embodiments 4 or 12-25, wherein the increased activity of the transaldolase is achieved by heterologously expressing a transaldolase.

Embodiment 27: The engineered microbial cell of embodiment 26, wherein the heterologous transaldolase includes a transaldolase from Corynebacterium glutamicum or Saccharomyces cerevisiae.

Embodiment 28: The engineered microbial cell of any one of embodiments 4 or 12-27, wherein the increased activity of the 3-dehydroquinate synthase is achieved by heterologously expressing a 3-dehydroquinate synthase.

Embodiment 29: The engineered microbial cell of embodiment 28, wherein the heterologous 3-dehydroquinate synthase includes a 3-dehydroquinate synthase from Corynebacterium glutamicum or Saccharomyces cerevisiae.

Embodiment 30: The engineered microbial cell of embodiment 29, wherein the heterologous 3-dehydroquinate synthase includes a 3-dehydroquinate synthase from Saccharomyces cerevisiae.

Embodiment 31: The engineered microbial cell of embodiment 30, wherein the heterologous 3-dehydroquinate synthase is from S. cerevisiae 288c (UniProt ID P08566) and includes SEQ ID NO:6, wherein, the engineered microbial cell also expresses: a 3-dehydroshikimate dehydratase from Neurospora crassa ATCC 24698 (UniProt ID P07046) including SEQ ID NO:1; a transaldolase from S. cerevisiae 288c (UniProt ID P53228) including SEQ ID NO:5; and/or an enolase from S. cerevisiae 288c (UniProt IDP00924) including SEQ ID NO:7.

Embodiment 32: The engineered microbial cell of any one of embodiments 8 or 16-31, wherein the feedback-deregulated DAHP synthase is a variant of a S. cerevisiae feedback-deregulated DAHP synthase.

Embodiment 33: The engineered microbial cell of embodiment 32, wherein the feedback-deregulated DAHP synthase is from S. cerevisiae (UniProt ID P32449), includes amino acid substitution K229L, and includes SEQ ID NO:3, wherein the engineered microbial cell also expresses: a 3-dehydroshikimate dehydratase from Neurospora crassa ATCC 24698 (UniProt ID P07046) including SEQ ID NO:1; a 3-dehydroshikimate dehydratase from C. glutamicum ATCC 13032 (UniProt ID O52377) including SEQ ID NO:9; and/or a transaldolase from C. glutamicum ATCC 13032 (UniProt ID Q8NQ64) including SEQ ID NO:8.

Embodiment 34: The engineered microbial cell of any one of embodiments 1-33, wherein, when cultured, the engineered microbial cell produces 3,4-dihydroxybenzoic acid at a level at least 350 mg/L of culture medium.

Embodiment 35: A culture of engineered microbial cells according to any one of embodiments 1-34.

Embodiment 36: The culture of embodiment 35, wherein the substrate includes a carbon source and a nitrogen source selected from the group consisting of urea, an ammonium salt, ammonia, and any combination thereof.

Embodiment 37: The culture of embodiment 35 or embodiment 36, wherein the engineered microbial cells are present in a concentration such that the culture has an optical density at 600 nm of 10-500.

Embodiment 38: The culture of any one of embodiments 35-37, wherein the culture includes 3,4-dihydroxybenzoic acid.

Embodiment 39: The culture of any one of embodiments 35-38, wherein the culture includes 3,4-dihydroxybenzoic acid at a level at least 350 ng/L of culture medium.

Embodiment 40: A method of culturing engineered microbial cells according to any one of embodiments 1-34, the method including culturing the cells under conditions suitable for producing 3,4-dihydroxybenzoic acid.

Embodiment 41: The method of embodiment 40, wherein the method includes fed-batch culture, with an initial glucose level in the range of 1-100 g/L, followed controlled sugar feeding.

Embodiment 42: The method of embodiment 40 or embodiment 41, wherein the fermentation substrate includes glucose and a nitrogen source selected from the group consisting of urea, an ammonium salt, ammonia, and any combination thereof.

Embodiment 43: The method of any one of embodiments 40-42, wherein the culture is pH-controlled during culturing.

Embodiment 44: The method of any one of embodiments 40-43, wherein the culture is aerated during culturing.

Embodiment 45: The method of any one of embodiments 40-44, wherein the engineered microbial cells produce 3,4-dihydroxybenzoic acid at a level at least 350 mg/L of culture medium.

Embodiment 46: The method of any one of embodiments 40-45, wherein the method additionally includes recovering 3,4-dihydroxybenzoic acid from the culture.

Embodiment 47: A method for preparing 3,4-dihydroxybenzoic acid using microbial cells engineered to produce 3,4-dihydroxybenzoic acid, the method including:
(a) expressing a non-native 3-dehydroshikimate dehydratase in microbial cells;
(b) cultivating the microbial cells in a suitable culture medium under conditions that permit the microbial cells to produce 3,4-dihydroxybenzoic acid, wherein the 3,4-dihydroxybenzoic acid is released into the culture medium; and (c) isolating 3,4-dihydroxybenzoic acid from the culture medium.

### BRIEF DESCRIPTION OF THE DRAWINGS

FIG. 1: Pathway for production of 3,4-dihydroxybenzoic acid (3,4-dihydroxybenzoate) by fermentation.

FIG. 2: 3,4-dihydroxybenzoate titers measured in the extracellular broth following fermentation of first-round-engineered host Saccharomyces cerevisiae.

FIG. 3: 3,4-dihydroxybenzoate titers measured in the extracellular broth following fermentation of second-round engineered host S. cerevisiae.

FIG. 4: A “split-marker, double-crossover” genomic integration strategy, which was developed to engineer S. cerevisiae strains. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F).

FIG. 5: A “loop-in, single-crossover” genomic integration strategy, which was developed to engineer C. glutamicum strains. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (e.g., 25 µg/ml kanamycin). Correct genomic integration in colonies derived from loop-in integration were confirmed by colony PCR with UF/IR and DR/IF PCR primers. Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome of C. glutamicum. Note: only one of two possible integrations is shown here. Correct genomic integration was confirmed by colony PCR and counter-selection was applied so that the plasmid backbone and counter-selection marker could be excised. This results in one of two possibilities: reversion to wild-type or the desired pathway integration. Again, correct genomic loop-out is confirmed by colony PCR. (Abbreviations: Primers: UF = upstream forward, DR = downstream reverse, IR = internal reverse, IF = internal forward.) See Example 1.

### DETAILED DESCRIPTION

The present disclosure describes the engineering of microbial cells for fermentative production of 3,4-dihydroxybenzoic acid and provides novel engineered microbial cells and cultures, as well as related 3,4-dihydroxybenzoic acid production methods.

### Definitions

Terms used in the claims and specification are defined as set forth below unless otherwise specified.

The term “fermentation” is used herein to refer to a process whereby a microbial cell converts one or more substrate(s) into a desired product (such as 3,4-dihydroxybenzoic acid) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “3,4-dihydroxybenzoic acid” refers to a chemical compound of the formula C₇H₆O₄, also known as “3,4-dihydroxybenzoate” (CAS# 99-50-3).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., 3,4-dihydroxybenzoic acid) produced by a culture of microbial cells divided by the culture volume.

As used herein with respect to recovering 3,4-dihydroxybenzoic acid from a cell culture, “recovering” refers to separating the 3,4-dihydroxybenzoic acid from at least one other component of the cell culture medium.

### Engineering Microbes for 3,4-Dihydroxybenzoic Acid Production

### 3,4-Dihydroxybenzoic Acid Biosynthesis Pathway

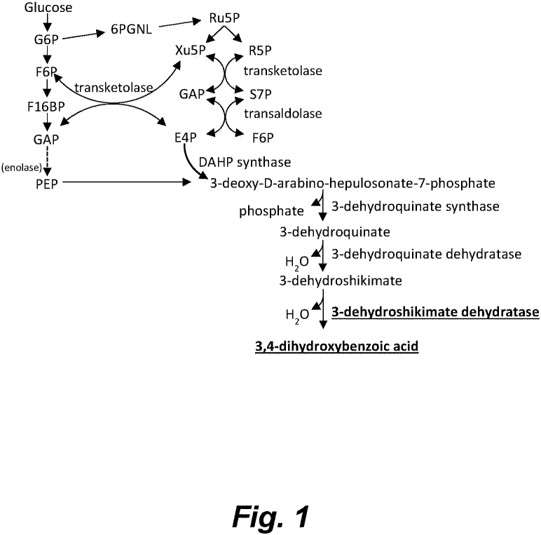

The metabolic pathway to 3,4-dihydroxybenzoic acid is derived from the shikimate pathway metabolite, 3-dehydroshikimate. (See FIG. 1.) The production of 3,4-dihydroxybenzoic acid by fermentation of a simple carbon source can be achieved by improving flux of the shikimate biosynthesis pathway and linking the flux to an active 3-dehydroshikimate dehydratase in a suitable microbial host. 3-dehydroshikimate is derived from the aromatic branch of amino acid biosynthesis, based on the precursors phosphoenolpyruvate (PEP) and erythrose-4-phosphate (E4P). The first step of the biosynthesis pathway (carried out by 3-deoxy-D-arabinoheptulosonate 7-phosphate [DAHP] synthase) is subject to feedback inhibition by the aromatic amino acids tyrosine, tryptophan, and phenylalanine. Production of 3,4-dihydroxybenzoic acid in Saccharomyces cerevisiae, for example, is enabled by the addition of a single heterologous enzymatic step, 3-dehydroshikimate dehydratase.

### Engineering for Microbial 3,4-Dihydroxybenzoic Acid Production

Any 3-dehydroshikimate dehydratase that is active in the microbial cell being engineered may be introduced into the cell, typically by introducing and expressing the gene(s) encoding the enzyme(s) using standard genetic engineering techniques. Suitable 3,4-dihydroxybenzoic acid synthases may be derived from any source, including plant, archaeal, fungal, gram-positive bacterial, and gram-negative bacterial sources (see, e.g., those described herein).

One or more copies of any of these genes can be introduced into a selected microbial host cell. If more than one copy of a gene is introduced, the copies can have the same or different nucleotide sequences. In some embodiments, one or both (or all) of the heterologous gene(s) is/ are expressed from a strong, constitutive promoter. In some embodiments, the heterologous gene(s) is/are expressed from an inducible promoter. The heterologous gene(s) can optionally be codon-optimized to enhance expression in the selected microbial host cell. The codon-optimization tables used in the Examples are as follows: Bacillus subtilis Kazusa codon table: www.kazusa.orjp/codon/cgi-bin/showcodon.cgi?species=1423&aa=1&style=N; Yarrowia lipolytica Kazusa codon table: www.kazusa.or.jp/codon/cgi-bin/showcodon.cgi?species-4952&aa=1&style=N; Corynebacteria glutamicum Kazusa codon table: www.kazusa.or.jp/codon/cgi-bin/showcodon.cgi?species=340322&aa=1&style=N;Saccharomyces cerevisiae Kazusa codon table: http://www.kazusa.orjp/codon/cgi-bin/showcodon.cgi?species=4932&aa=1&style=N.Also used, was a modified, combined codon usage scheme for S. cereviae and C. glutamicum, which is reproduced below.

Modified Codon Usage Table for Sc and Cg

| Amino Acid | Codon | Fraction |
|---|---|---|
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GAA | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | AAG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | AAT | 0.33 |
| N | AAC | 0.67 |
| P | CCG | 0.22 |
| P | CCA | 0.35 |
| P | CCT | 0.23 |
| P | CCC | 0.2 |
| Q | CAG | 0.61 |
| Q | CAA | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| w | TGG | 1 |
| Y | TAT | 0.34 |
| Y | TAC | 0.66 |

### Increasing the Activity of Upstream Enzymes

One approach to increasing 3,4-dihydroxybenzoic acid production in a microbial cell that is capable of such production is to increase the activity of one or more upstream enzymes in the 3,4-dihydroxybenzoic acid biosynthesis pathway. Upstream pathway enzymes include all enzymes involved in the conversions from a feedstock all the way to a metabolite that can be directly converted to 3,4-dihydroxybenzoic acid (e.g., 3-dehydroshikimate). Illustrative enzymes, for this purpose, include, but are not limited to, those shown in FIG. 1 in the pathway leading to this metabolite. Suitable upstream pathway genes encoding these enzymes may be derived from any available source, including, for example, those disclosed herein.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

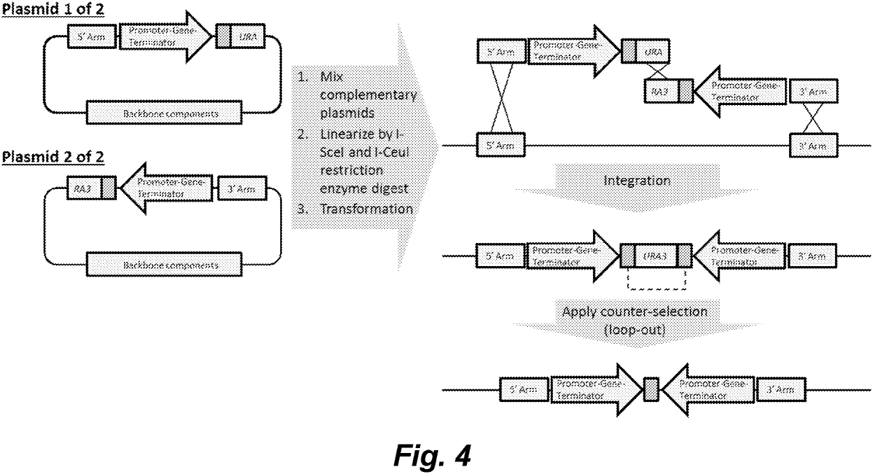

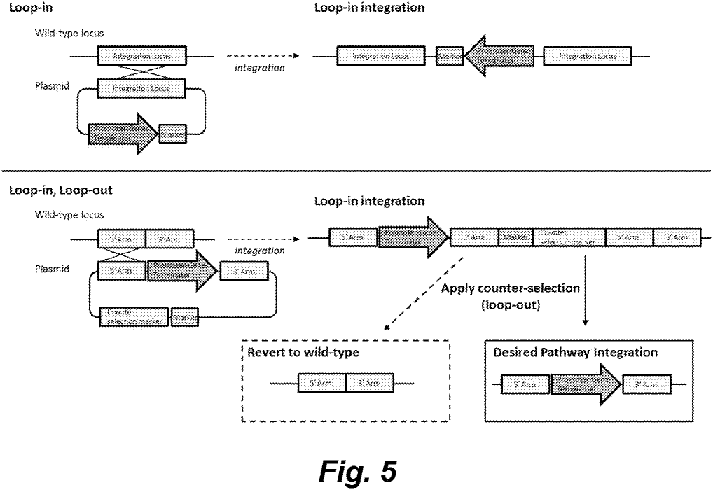

Alternatively, or in addition, one or more promoters can be substituted for native promoters using, for example, a technique such as that illustrated in FIG. 4. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of 3,4-dihydroxybenzoic acid production and expressed from a strong constitutive promoter and/or can optionally be codon-optimized to enhance expression in the selected microbial host cell.

In various embodiments, the engineering of a 3,4-dihydroxybenzoic acid-producing microbial cell to increase the activity of one or more upstream pathway enzymes increases the 3,4-dihydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3,4-dihydroxybenzoic acid titer is in the range of 10-fold to 1000-fold, 20-fold to 500-fold, 50-fold to 400-fold, 10-fold to 300-fold, or any range bounded by any of the values listed above. (Ranges herein include their endpoints.) These increases are determined relative to the 3,4-dihydroxybenzoic acid titer observed in a 3,4-dihydroxybenzoic acid-producing microbial cell that lacks any increase in activity of upstream pathway enzymes. This reference cell may have one or more other genetic alterations aimed at increasing 3,4-dihydroxybenzoic acid production.

In various embodiments, the 3,4-dihydroxybenzoic acid titers achieved by increasing the activity of one or more upstream pathway enzymes are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

### Introduction of Feedback-Deregulated Enzymes

Since aromatic amino acid biosynthesis is subject to feedback inhibition, another approach to increasing 3,4-dihydroxybenzoic acid production in a microbial cell engineered to express a heterologous 3-dehydroshikimate dehydratase is to introduce feedback-deregulated forms of one or more enzymes that are normally subject to feedback inhibition in the 3-dehydroshikimate dehydratase-expressing microbial cell. DAHP synthase is an example of such an enzyme. A feedback-deregulated form can be a heterologous, wild-type enzyme that is less sensitive to feedback inhibition than the endogenous enzyme in the particular microbial host cell. Alternatively, a feedback-deregulated form can be a variant of an endogenous or heterologous enzyme that has one or more mutations rendering it less sensitive to feedback inhibition than the corresponding wild-type enzyme. Examples of the latter include variant DAHP synthases (two from S. cerevisiae, one from E. coli) that have known point mutations rendering them resistant to feedback inhibition, e.g., S. cerevisiae ARO4Q166K, S. cerevisiae AR04K229L, and E. coli AroGD146N. The last 5 characters of these designations indicate amino acid substitutions, using the standard one-letter code for amino acids, with the first letter referring to the wild-type residue and the last letter referring to the replacement reside; the numbers indicate the position of the amino acid substitution in the translated protein.

In various embodiments, the engineering of a 3-dehydroshikimate dehydratase-expressing microbial cell to express a feedback-deregulated enzymes increases the 3,4-dihydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in 3,4-dihydroxybenzoic acid titer is in the range of 10 percent to 100-fold, 2-fold to 50-fold, 5-fold to 40-fold, 10-fold to 30-fold, or any range bounded by any of the values listed above. These increases are determined relative to the 3,4-dihydroxybenzoic acid titer observed in a 3,4-dihydroxybenzoic acid-producing microbial cell that does not express a feedback-deregulated enzyme. This reference cell may (but need not) have other genetic alterations aimed at increasing 3,4-dihydroxybenzoic acid production, i.e., the cell may have increased activity of an upstream pathway enzyme resulting from some means other than feedback-insensitivity.

In various embodiments, the 3,4-dihydroxybenzoic acid titers achieved by using a feedback-deregulated enzyme to increase flux though the 3,4-dihydroxybenzoic acid biosynthetic pathway are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

The approaches of supplementing the activity of one or more endogenous enzymes and/or introducing one or more feedback-deregulated enzymes can be combined in 3-dehydroshikimate dehydratase-expressing microbial cells to achieve even higher 3,4-dihydroxybenzoic acid production levels.

### Reduction of Consumption of 3,4-Dihydroxybenzoic Acid and/or Its Precursors

Another approach to increasing 3,4-dihydroxybenzoic acid production in a microbial cell that is capable of such production is to decrease the activity of one or more enzymes that consume one or more 3,4-dihydroxybenzoic acid pathway precursors, such as enzymes that produce the amino acids tyrosine, phenylalanine and tryptophan. An example is the enzyme activity EC 1.1.1.25 (which has multiple enzyme names: shikimate dehydrogenase; dehydroshikimic reductase; shikimate oxidoreductase; shikimate:NADP+ oxidoreductase; 5-dehydroshikimate reductase; shikimate 5-dehydrogenase; 5-dehydroshikimic reductase; DHS reductase; shikimate:NADP+ 5-oxidoreductase; AroE), or the systematic name shikimate:NADP+ 3-oxidoreductase. In Saccharomyces, the activity is found in the pentafunctional AROM polypeptide, called ARO1. This is the enzyme step that converts (commits) the 3,4-dihydrooxybenzoic acid pathway intermediate 3-dehydroshikimate to aromatic amino acid biosynthesis. Decreasing the activity of that enzyme reaction in ARO1 would be beneficial to producing 3,4-dihydrooxybenzoic acid. In some embodiments, the activity of one or more such enzymes is reduced by modulating the expression or activity of the native enzyme(s). The activity of such enzymes can be decreased, for example, by substituting the native promoter of the corresponding gene(s) with a less active or inactive promoter or by deleting the corresponding gene(s).

In various embodiments, the engineering of a 3,4-dihydroxybenzoic acid-producing microbial cell to reduce precursor consumption by one or more side pathways increases the 3,4-dihydroxybenzoic acid titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold. In various embodiments, the increase in 3,4-dihydroxybenzoic acid titer is in the range of 10-fold to 1000-fold, 20-fold to 500-fold, 50-fold to 400-fold, 10-fold to 300-fold, or any range bounded by any of the values listed above. These increases are determined relative to the 3,4-dihydroxybenzoic acid titer observed in a 3,4-dihydroxybenzoic acid-producing microbial cell that does not include genetic alterations to reduce precursor consumption. This reference cell may (but need not) have other genetic alterations aimed at increasing 3,4-dihydroxybenzoic acid production, i.e., the cell may have increased activity of an upstream pathway enzyme.

In various embodiments, the 3,4-dihydroxybenzoic acid titers achieved by reducing precursor consumption are at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 850 mg/L, 100 mg/L to 800 mg/L, 200 mg/L to 750 mg/L, 250 mg/L to 700 mg/L, 300 mg/L to 650 mg/L, 350 mg/L to 600 mg/L, or any range bounded by any of the values listed above.

Any of the approaches for increasing 3,4-dihydroxybenzoic acid production described above can be combined, in any combination, to achieve even higher 3,4-dihydroxybenzoic acid production levels.

### Illustrative Amino Acid and Nucleotide Sequences

The following table identifies amino acid and nucleotide sequences used in Example 1. The corresponding sequences are shown in the Sequence Listing.

SEO ID NO Cross-Reference Table

| SEQ ID NO: | E1 Uniprot ID | Enzyme 1 - activity name | E1 Modifications | Enzyme 1 - source organism |
|---|---|---|---|---|
| Amino Acid Sequences | | | | |
| 1 | P07046 | 3-dehydroshikimate dehydratase | | Neurospora crassa ATCC 24698 |
| 2 | I1RNW1 | 3-dehydroshikimate dehydratase | | Gibberella zeae strain PH-1 |
| 3 | P32449 | DAHP synthase | K229L,reduces pathway feedback inhibition | Saccharomyces cerevisiae S288c |
| 4 | Q9X5D2 | 3-dehydroquinate synthase | | Corynebacterium glutamicum ATCC 13032 |
| 5 | P53228 | Transaldolase | | Saccharomyces cerevisiae S288c |
| 6 | P08566 | 3-dehydroquinate synthase, 3-phosphoshikimate 1-carboxyvinyltransferase,3-phosphoshikimate 1-carboxyvinyltransferase,Shikimate kinase (SK),Shikimate 5-dehydrogenase,3-dehydroquinate dehydratase (3-dehydroquinase) | | Saccharomyces cerevisiae S288c |
| 7 | P00924 | Enolase | | Saccharomyces cerevisiae S288c |
| 8 | Q8NQ64 | Transaldolase | | Corynebacterium glutamicum ATCC 13032 |
| 9 | 052377 | 3-dehydroquinate dehydratase (3-dehydroquinase) | | Corynebacterium glutamicum ATCC 13032 |
| DNA Sequences | | | | |
| 10 | P07046 | 3-dehydroshikimate dehydratase | | Neurospora crassa ATCC 24698 |
| 11 | I1RNW1 | 3-dehydroshikimate dehydratase | | Gibberella zeae strain PH-1 |
| 12 | P32449 | DAHP synthase | K229L,reduces pathway feedback inhibition | Saccharomyces cerevisiae S288c |
| 13 | Q9X5D2 | 3-dehydroquinate synthase | | Corynebacterium glutamicum ATCC 13032 |
| 14 | P53228 | Transaldolase | | Saccharomyces cerevisiae S288c |
| 15 | P08566 | 3-dehydroquinate synthase,3-phosphoshikimate 1-carboxyvinyltransferase,3-phosphoshikimate 1-carboxyvinyltransferase,Shikimate kinase (SK),Shikimate 5-dehydrogenase,3-dehydroquinate dehydratase (3-dehydroquinase) | | Saccharomyces cerevisiae S288c |
| 16 | P00924 | Enolase | | Saccharomyces cerevisiae S288c |
| 17 | Q8NQ64 | Transaldolase | | Corynebacterium glutamicum ATCC 13032 |
| 18 | 052377 | 3-dehydroquinate dehydratase (3-dehydroquinase) | | Corynebacterium glutamicum ATCC 13032 |

### Microbial Host Cells

Any microbe that can be used to express introduced genes can be engineered for fermentative production of 3,4-dihydroxybenzoic acid as described above. In certain embodiments, the microbe is one that is naturally incapable of fermentative production of 3,4-dihydroxybenzoic acid. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B.clausii, B. halodurans, B. megaterium, B. coagulans, B. circulans, B. lautus, B. thuringiensis, S. albus, S.lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T. reesei, T. viride, F. oxysporum, or F. solani. Illustrative plasmids or plasmid components for use with such hosts include those described in U.S. Pat. Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Pat. Pub. No. 2011/0045563.

In some embodiments, the host cell can be an algal cell derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Pat. Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Pat. Pub. Nos. 2010/0297749 and 2009/0282545 and in Intl. Pat. Pub. No. WO 2011/034863.

### Genetic Engineering Methods

Microbial cells can be engineered for fermentative 3,4-dihydroxybenzoic acid production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Pat. Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F.A., et al., (“In vivo genome editing using Staphylococcus aureus Cas9,” Nature 520(7546): 186-91, 2015, Apr 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Pat. Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, 3,4-dihydroxybenzoic acid. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for 3,4-dihydroxybenzoic acid production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous (e.g., non-native) gene, e.g., a 3-dehydroshikimate dehydratase gene. In various embodiments, the microbial cell can include and express, for example: (1) a single 3-dehydroshikimate dehydratase gene, (2) two or more heterologous 3-dehydroshikimate dehydratase genes, which can be the same or different (in other words, multiple copies of the same heterologous 3-dehydroshikimate dehydratase gene can be introduced or multiple, different heterologous 3-dehydroshikimate dehydratase genes can be introduced), (3) a single heterologous 3-dehydroshikimate dehydratase gene that is not native to the cell and one or more additional copies of an native 3-dehydroshikimate dehydratase gene (if applicable), or (4) two or more non-native 3-dehydroshikimate dehydratase genes, which can be the same or different, and one or more additional copies of a native 3-dehydroshikimate dehydratase gene (if applicable).

In certain embodiments, this engineered host cell can include at least one additional genetic alteration that increases flux through any pathway leading to the production of an immediate precursor of 3,4-dihydroxybenzoic acid. As discussed above, this can be accomplished by one or more of the following: increasing the activity of upstream enzymes, e.g., by introducing a feedback-deregulated version of a DAHP synthase, alone or in combination with other means for increasing the activity of upstream enzymes.

The engineered microbial cells can contain introduced genes that have a native nucleotide sequence or that differ from native. For example, the native nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in yeast cells, namely S. cerevisiae. (See Example 1.)

### Illustrative Engineered Yeast Cells

In certain embodiments, the engineered yeast (e.g., S. cerevisiae) cell expresses one or more non-native 3-dehydroshikimate dehydratase(s) having at least 70 percent, 75 percent, 80 percent, 85 percent, 90 percent, 95 percent or 100 percent amino acid sequence identity with a 3-dehydroshikimate dehydratase from Neurospora crassa ATCC 24698 (UniProt ID P07046); and/or one or more non-native 3-dehydroshikimate dehydratase(s) having at least 70 percent, 75 percent, 80 percent, 85 percent, 90 percent, 95 percent or 100 percent amino acid sequence identity with a 3-dehydroshikimate dehydratase from C. glutamicum ATCC 13032 (UniProt ID O52377); and/or one or more feedback-deregulated DAHP synthase(s) having at least 70 percent, 75 percent, 80 percent, 85 percent, 90 percent, 95 percent or 100 percent amino acid sequence identity with a feedback-deregulated DAHP synthase from S. cerevisiae (UniProt ID P32449), harboring amino acid substitution K229L; and/or one or more heterologous transaldolase(s) having at least 70 percent, 75 percent, 80 percent, 85 percent, 90 percent, 95 percent or 100 percent amino acid sequence identity with a transaldolase from C. glutamicum ATCC 13032 (UniProt ID Q8NQ64).

In particular embodiments:
the 3-dehydroshikimate dehydratase from Neurospora crassa ATCC 24698 (UniProt ID P07046) includes SEQ ID NO: 1;
the 3-dehydroshikimate dehydratase from C. glutamicum ATCC 13032 (UniProt ID O52377) includes SEQ ID NO:9;
the feedback-deregulated DAHP synthase from S. cerevisiae (UniProt ID P32449), harboring amino acid substitution K229L, includes SEQ ID NO:3; and/or
the transaldolase from C. glutamicum ATCC 13032 (UniProt ID Q8NQ64) includes SEQ ID NO:8.

In an illustrative embodiment, a titer of about 360 mg/L was achieved after engineering S. cerevisiae to express the 3-dehydroshikimate dehydratase from Neurospora crassa ATCC 24698 (UniProt ID P07046), the 3-dehydroshikimate dehydratase from C. glutamicum ATCC 13032(UniProt ID O52377), the feedback-deregulated DAHP synthase from S. cerevisiae (UniProt ID P32449), harboring amino acid substitution K229L, and the transaldolase from C. glutamicum ATCC 13032 (UniProt ID Q8NQ64).

In other embodiments, the engineered yeast (e.g., S. cerevisiae) cell expresses one or more non-native 3-dehydroshikimate dehydratase(s) having at least 70 percent, 75 percent, 80 percent, 85 percent, 90 percent, 95 percent or 100 percent amino acid sequence identity with a 3-dehydroshikimate dehydratase from Neurospora crassa ATCC 24698 (UniProt ID P07046); and/or one or more heterologous 3-dehydroquinate synthase(s) having at least 70 percent, 75 percent, 80 percent, 85 percent, 90 percent, 95 percent or 100 percent amino acid sequence identity with a 3-dehydroquinate synthase from S. cerevisiae 288c (UniProt ID P08566); and/or one or more heterologous transaldolase(s) having at least 70 percent, 75 percent, 80 percent, 85 percent, 90 percent, 95 percent or 100 percent amino acid sequence identity with a transaldolase from S. cerevisiae 288c (UniProt ID P53228); and/or one or more heterologous enolase(s) having at least 70 percent, 75 percent, 80 percent, 85 percent, 90 percent, 95 percent or 100 percent amino acid sequence identity with an enolase from S. cerevisiae 288c (UniProt IDP00924).

In particular embodiments:
the 3-dehydroshikimate dehydratase from Neurospora crassa ATCC 24698 (UniProt ID P07046) includes SEQ ID NO: 1;
the 3-dehydroquinate synthase from S. cerevisiae 288c (UniProt ID P08566) includes SEQ ID NO:6;
the transaldolase from S. cerevisiae 288c (UniProt ID P53228) includes SEQ ID NO:5; and/or
the enolase from S. cerevisiae 288c (UniProt IDP00924) includes SEQ ID NO:7.

In an illustrative embodiment, a titer of about 520 mg/L was achieved after engineering S. cerevisiae to express the 3-dehydroshikimate dehydratase from Neurospora crassa ATCC 24698 (UniProt ID P07046), the 3-dehydroquinate synthase from S. cerevisiae 288c (UniProt ID P08566), the transaldolase from S. cerevisiae 288c (UniProt ID P53228), and the enolase from S. cerevisiae 288c (UniProt IDP00924).

### Culturing of Engineered Microbial Cells

Any of the microbial cells described herein can be cultured, e.g., for maintenance, growth, and/or 3,4-dihydroxybenzoic acid production.

In some embodiments, the cultures are grown to an optical density at 600 nm of 10-500, such as an optical density of 50-150.

In various embodiments, the cultures include produced 3,4-dihydroxybenzoic acid at titers of at least 10, 20, 30, 40, 50, 75, 100, 150, 200, 250, 300, 350, 400, 450, 500, 550, 600, 650, 700, 750, 800, 850, or 900 mg/L or at least 1, 1.5, 2, 2.5, 3, 3.5, 4, 4.5, 5, 10, 15, 20, 25 gm/L. In various embodiments, the titer is in the range of 50 mg/L to 900 mg/L, 75 mg/L to 800 mg/L, 100 mg/L to 700 mg/L, 200 mg/L to 600 mg/L, 250 mg/L to 500 mg/L, 300 mg/L to 450 mg/L, 350 mg/L to 400 mg/L or any range bounded by any of the values listed above.

### Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce 3,4-dihydroxybenzoic acid, the culture medium can include, and/or is supplemented during culture with, glucose and/or a nitrogen source such as urea, an ammonium salt, ammonia, or any combination thereof.

### Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C. -75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40 % (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70 % (w/v), 20-60 % (w/v), or 30-50 % (w/v). Furthermore, different sugar levels can be used for different phases of culturing. For fed-batch culture (e.g., of S. cerevisiae or C. glutamicum), the sugar level can be about 100-200 g/L (10-20 % (w/v)) in the batch phase and then up to about 500-700 g/L (50-70 % in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3 % (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

### 3,4-Dihydroxybenzoic Acid Production and Recovery

Any of the methods described herein may further include a step of recovering 3,4-dihydroxybenzoic acid. In some embodiments, the produced 3,4-dihydroxybenzoic acid contained in a so-called harvest stream is recovered/harvested from the production vessel. The harvest stream may include, for instance, cell-free or cell-containing aqueous solution coming from the production vessel, which contains 3,4-dihydroxybenzoic acid as a result of the conversion of production substrate by the resting cells in the production vessel. Cells still present in the harvest stream may be separated from the 3,4-dihydroxybenzoic acid by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced 3,4-dihydroxybenzoic acid from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify 3,4-dihydroxybenzoic acid. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### EXAMPLE 1 - CONSTRUCTION AND SELECTION OF STRAINS OF SACCHAROMYCES CEREVISIAE AND CORYNEBACTERIUM GLUTAMICUM ENGINEERED TO PRODUCE 3,4-DIHYDROXYBENZOIC ACID

### Plasmid/DNA Design

All strains tested for this work were transformed with plasmid DNA designed using proprietary software. Plasmid designs were specific to one of the two host organisms engineered in this work. The plasmid DNA was physically constructed by a standard DNA assembly method. This plasmid DNA was then used to integrate metabolic pathway inserts by one of two host-specific methods, each described below.

### S. Cerevisiae Pathway Integration

A “split-marker, double-crossover” genomic integration strategy has been developed to engineer S. cerevisiae strains. FIG. 2 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and reconstituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knockout, gene knock-in, and promoter titration in the same workflow.

### C. Glutamicum Pathway Integration

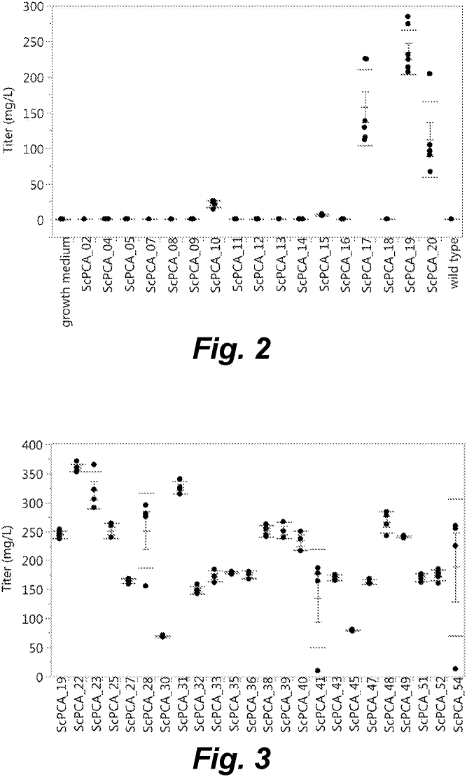

A “loop-in, single-crossover” genomic integration strategy has been developed to engineer C. glutamicum strains. FIG. 3 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (25 µg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome of C. glutamicum. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF = upstream forward, DR = downstream reverse, IR = internal reverse, IF = internal forward.)

### Cell Culture

Separate workflows were established for C. glutamicum and S. cerevisiae due to differences in media requirements and growth. Both processes involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (BHI for C. glutamicum, SD-ura for S. cerevisiae) and cultivated for two days until saturation and then frozen with 16.6% glycerol at -80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

### Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

### Cell Viability

Two methods were used to measure cell viability. The first assay utilized a single stain, propidium iodide, to assess cell viability. Propidium iodide binds to DNA and is permeable to cells with compromised cell membranes. Cells that take up the propidium iodide are considered non-viable. A dead cell control was used to normalize to total number of cells, by incubating a cell sample of control culture at 95° C. for 10 minutes. These control samples and test samples were incubated with the propidium iodide stain for 5 minutes, washed twice with 175 mM phosphate buffer, and fluorescence measured in black solid-bottom 96-well plates at 617 nm.

### Glucose

Glucose is measured using an enzymatic assay with 16 U/mL glucose oxidase (Sigma) with 0.2 U/mL horseradish peroxidase (Sigma) and 0.2 mM Amplex red in 175 mM sodium phosphate buffer, pH 7. Oxidation of glucose generates hydrogen peroxide, which is then oxidized to reduce Amplex red, which changes absorbance at 560 nm. The change is absorbance is correlated to the glucose concentration in the sample using standards of known concentration.

### Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 µL of supernatant was transferred to each plate, with one stored at 4° C., and the second stored at 80° C. for long-term storage.

### Genetic Engineering Approach and Results

A library approach was taken to identify functional enzymes in the host organism, which was Saccharomyces cerevisiae. A broad search of 3-dehydroshikimate dehydratase sequences identified in total 17 orthologous sequences from these sources: 6 fungi and 11 bacterial. The 3-dehydroshikimate dehydratase enzymes were codon-optimized and expressed in S. cerevisiae.

### First Round of Engineering

Titer was achieved in S. cerevisiae strains in the initial proof-of-concept experiments. In particular, 240 mg/L titer was produced in the first round of engineering by integration of 3-dehydroshikimate dehydratase (UniProt ID P07046) from Neurospora crassa ATCC 24698. The 3-dehydroshikimate dehydratase from Bacillus anthracis (UniProt ID Q81RQ4), Colletotrichum fioriniae PJ7 (UniProt ID A0A010RUW7), and Gibberella zeae strain PH-1 (UniProt ID I1RNW1) are also active in S. cerevisiae and enabled production of 20-150 mg/L 3,4-dihydroxybenzoic acid. (See FIG. 2.)

TABLE 1

First-Round Results

| Strain Name | Titer (µg/L) | E1 Uniprot ID | Enzyme 1 - activity name | Enzyme 1 - source organism | E1 Codon Optimization Abbrev. |
|---|---|---|---|---|---|
| ScPCA_ 02 | 204.6 | A0A117EE17 | 3-dehydroshikimate dehydratase | Streptomyces scabiei | modified Cg codon usage |
| ScPCA_ 04 | 201.1 | A0A0F0LTK0 | 3-dehydroshikimate dehydratase | Microbacterium azadirachtae | modified Cg codon usage |
| ScPCA_ 05 | 180.8 | A0A0K8QDN7 | 3-dehydroshikimate dehydratase | Arthrobacter sp. Hiyo1 | modified Cg codon usage |
| ScPCA_ 07 | 181.4 | X5LJS7 | 3-dehydroshikimate dehydratase | Mycobacterium mageritense DSM 44476 | modified Cg codon usage |
| ScPCA_ 08 | 172.8 | A0A0H4R9P8 | 3-dehydroshikimate dehydratase | Bacillus megaterium Q3 | modified Cg codon usage |
| ScPCA_ 09 | 187.6 | Q43922 | 3-dehydroshikimate dehydratase | Acinetobacter baylyi ATCC 33305 | modified Cg codon usage |
| ScPCA_ 10 | 21834.7 | Q81 RQ4 | 3-dehydroshikimate dehydratase | Bacillus anthracis | modified Cg codon usage |
| ScPCA_ 11 | 166.6 | Q6W2E1 | 3-dehydroshikimate dehydratase | Sinorhizobium fredii NBRC 101917 | modified Cg codon usage |
| ScPCA_ 12 | 240.6 | A0A031H2V8 | 3-dehydroshikimate dehydratase | Stenotrophomonas sp. RIT309 | modified Cg codon usage |
| ScPCA_ 13 | 199.3 | D0S8E6 | 3-dehydroshikimate dehydratase | Acinetobacter johnsonii SH046 | modified Cg codon usage |
| ScPCA_ 14 | 171.0 | A0A0A80XR8 | 3-dehydroshikimate dehydratase | Xanthomonas sacchari | modified Cg codon usage |
| ScPCA_ 15 | 6178.9 | N4UVG2 | 3-dehydroshikimate dehydratase | Fusarium oxysporum f. sp. cubense strain race 1 | modified Cg codon usage |
| ScPCA_ 16 | 196.1 | Q4WMT9 | 3-dehydroshikimate dehydratase | Neosartorya fumigata ATCC MYA-4609 | modified Cg codon usage |
| ScPCA_ 17 | 157399.5 | A0A010RUW7 | 3-dehydroshikimate dehydratase | Colletotrichum fioriniae PJ7 | modified Cg codon usage |
| ScPCA_ 18 | 171.4 | C5JZK4 | 3-dehydroshikimate dehydratase | Ajellomyces dermatitidis SLH14081 | modified Cg codon usage |
| ScPCA_ 19 | 240251.4 | P07046 | 3-dehydroshikimate dehydratase | Neurospora crassa ATCC 24698 | modified Cg codon usage |
| ScPCA_ 20 | 124720.2 | I1RNW1 | 3-dehydroshikimate dehydratase | Gibberella zeae strain PH-1 | modified Cg codon usage |

### Second Round of Engineering

We introduced additional genetic changes into the best-performing S. cerevisiae strain improve production of 3,4-dihydroxybenzoic acid. We took a combinatorial library approach to introduce an additional copy of 1-3 upstream pathway genes (in addition to 3-dehydroshikimate dehydratase [UniProt ID P07046] from Neurospora crassa ATCC 24698), in separate daughter strains, under the control of a strong, constitutive promoter (Table 2). Upstream pathway genes represent all genes involved in the conversion of key precursors (i.e. E4P & PEP) into the last native metabolite (e.g., 3-dehydroshikimate) in the pathway leading to 3,4-dihydroxybenzoic acid. Enzymes successfully built into strains and tested in the combinatorial library approach are shown in the 3,4-dihydroxybenzoic acid pathway diagram (FIG. 1).

The most improved strain from the second round of engineering contained DAHP synthase (UniProt ID 32449) from S. cerevisiae, containing the amino acid substitution K229L, which reduces pathway feedback-inhibition.

Additional strains having improved titer were identified in the second round. One strain contained: 3-dehydroquinate synthase (UniProt ID Q9X5D2) from Corynebacterium glutamicum ATCC 13032, DAHP synthase (UniProt ID P32449) from S. cerevisiae, containing the amino acid substitution K229L, and 3-dehydroquinate dehydratase (3-dehydroquinase) (UniProt ID O52377) from C. glutamicum ATCC 13032. Another improved strain from the second round contained: 3-dehydroquinate synthase (UniProt ID Q9X5D2) from C. glutamicum ATCC 13032, DAHP synthase (UniProt ID P32449) from S. cerevisiae, containing the amino acid substitution K229L, and transaldolase (UniProt ID Q8NQ64) from C. glutamicum ATCC 13032.

In addition to expressing additional upstream pathway enzymes, to further improve 3,4-dihydroxybenzoic acid production in S. cerevisiae, it is anticipated that replacing the native promoters of enzymes that consume 3,4-dihydroxybenzoic acid pathway metabolites (e.g., enzymes to make amino acids tyrosine, phenylalanine and tryptophan) to lower the activity of these enzymes will be beneficial.

The strains in the table below also contain the best enzyme identified in first round: 3-dehydroshikimate dehydratase (UniProt P07046). In addition, the DAHP synthase (UniProt ID P32449, from Saccharomyces cerevisiae) tested in the second round of strain engineering contained K229L to reduce pathway feedback-inhibition.

TABLE 2

Second-Round Results

| Strain Name | Titer (mg/L) | E1 Uniprot ID | Enzyme 1 - activity name | Enzyme 1 - source organism | E1 Codon Optimization Abbrev. | E2 Uniprot ID | Enzyme 2 -activity name | Enzyme 2 - source organism | E2 Codon Optimization Abbrev. | E3 Uniprot ID | Enzyme 3 - activity name | Enzyme 3 - source organism | E3 Codon Optimization Abbrev. |
|---|---|---|---|---|---|---|---|---|---|---|---|---|---|
| ScPC A_22 | 359.69 | P32449 | DAHP synthase | Saccharomyces cerevisiae S288c | Cg | Q8NQ64 | Transaldolase | Corynebacterium glutamicum ATCC 13032 | modified codon usage for Cg and Sc | O52 377 | 3-dehydroquinate dehydratase (3-dehydroquinase) | Corynebacterium glutamicum ATCC 13032 | modified codon usage for Cg and Sc |
| ScPC A_23 | 320.82 | Q9X5D2 | 3-dehydroquinate synthase | Corynebacterium glutamicum ATCC 13032 | modified codon usage for Cg and Sc | P32449 | DAHP synthase | Saccharomyces cerevisiae S288c | Corynebacterium glutamicum | O52 377 | 3-dehydroquinate dehydratase (3-dehydroquinase) | Corynebacterium glutamicum ATCC 13032 | modified codon usage for Cg and Sc |
| ScPC A_25 | 251.03 | Q9X5D2 | 3-dehydroquinate synthase | Corynebacterium glutamicum ATCC 13032 | modified codon usage for Cg and Sc | Q8NRS1 | Enolase | Corynebacterium glutamicum ATCC 13032 | modified codon usage for Cg and Sc | P32 449 | DAHP synthase | Saccharomyces cerevisiae S288c | Cg |
| ScPC A_27 | 165.34 | P53228 | Transaldolase | Saccharomyces cerevisiae S288c | modified codon usage for Cg and Sc | P53228 | Transaldolase | Saccharomyces cerevisiae S288c | modified codon usage for Cg and Sc | P53 228 | Transaldolase | Saccharomyces cerevisiae S288c | modified codon usage for Cg and Sc |
| ScPC A_28 | 251.78 | Q8NRS1 | Enolase | Corynebacterium glutamicum ATCC 13032 | modified codon usage for Cg and Sc | P32449 | DAHP synthase | Saccharomyces cerevisiae S288c | Corynebacterium glutamicum | O52 377 | 3-dehydroquinate dehydratase (3-dehydroquinase) | Corynebacterium glutamicum ATCC 13032 | modified codon usage for Cg and Sc |
| ScPC A_30 | 520.46 | P08566 | 3-dehydroquinate synthase,3-phosphoshikimate 1-carboxy-vinyltransferase,3-phosphoshikimate 1-carboxyvinyl-transferase,Shikimate kinase (SK),Shikimate 5-dehydrogenase,3-dehydroquinate dehydratase (3-dehydroquinase) | Saccharomyces cerevisiae S288c | modified codon usage for Cg and Sc | P53228 | Transaldolase | Saccharomyces cerevisiae S288c | modified codon usage for Cg and Sc | P00 924 | Enolase | Saccharomyces cerevisiae S288c | modified codon usage for Cg and Sc |
| ScPC A_31 | 325.80 | Q9X5D2 | 3-dehydroquinate synthase | Corynebacterium glutamicum ATCC 13032 | modified codon usage for Cg and Sc | P32449 | DAHP synthase | Saccharomyces cerevisiae S288c | Corynebacterium glutamicum | Q8 NQ 64 | Transaldolase | Corynebacterium glutamicum ATCC 13032 | modified codon usage for Cg and Sc |
| ScPC A_32 | 149.33 | P08566 | 3-dehydroquinate synthase,3-phosphoshikimate 1-carboxy-vinyltransferase,3-phosphoshikimate 1-carboxyvinyl-transferase,Shikimate kinase (SK),Shikimate 5-dehydrogenase,3-dehydroquinate dehydratase (3-dehydroquinase) | Saccharomyces cerevisiae S288c | modified codon usage for Cg and Sc | P00924 | Enolase | Saccharomyces cerevisiae S288c | modified codon usage for Cg and Sc | P32 449 | DAHP synthase | Saccharomyces cerevisiae S288c | Cg |
| ScPC A_33 | 172.52 | P00924 | Enolase | Saccharo-myces cerevisiae S288c | modified codon usage for Cg and Sc | P009 24 | Enolase | Saccharo-myces cerevisiae S288c | modified codon usage for Cg and Sc | P00 924 | Enolase | Saccharo-myces cerevisiae S288c | modified codon usage for Cg and Sc |
| ScPC A_35 | 179.21 | Q8NQ64 | Transaldolase | Corynebacterium glutamicum ATCC 13032 | modified codon usage for Cg and Sc | Q8NQ64 | Transaldolase | Corynebacterium glutamicum ATCC 13032 | modified codon usage for Cg and Sc | Q8 NQ 64 | Transaldolase | Corynebacterium glutamicum ATCC 13032 | modified codon usage for Cg and Sc |
| ScPC A_36 | 137.99 | Q9X5D2 | 3-dehydroquinate synthase | Corynebacterium glutamicum ATCC 13032 | modified codon usage for Cg and Sc | Q8NQ64 | Transaldolase | Corynebacterium glutamicum ATCC 13032 | modified codon usage for Cg and Sc | O52 377 | 3-dehydroquinate dehydratase (3-dehydroquinase) | Corynebacterium glutamicum ATCC 13032 | modified codon usage for Cg and Sc |
| ScPC A_38 | 250.35 | P53228 | Transaldolase | Saccharomyces cerevisiae S288c | modified codon usage for Cg and Sc | P32449 | DAHP synthase | Saccharomyces cerevisiae S288c | Corynebacterium glutamicum | P32 449 | DAHP synthase | Saccharomyces cerevisiae S288c | Cg |
| ScPC A_39 | 210.71 | P00924 | Enolase | Saccharomyces cerevisiae S288c | modified codon usage for Cg and Sc | P32449 | DAHP synthase | Saccharomyces cerevisiae S288c | Corynebacterium glutamicum | P32 449 | DAHP synthase | Saccharomyces cerevisiae S288c | Cg |
| ScPC A_40 | 176.85 | P14843 | Phospho-2-dehydro-3-deoxyheptonate aldolase | Saccharomyces cerevisiae S288c | modified codon usage for Cg and Sc | P14843 | Phospho-2-dehydro-3-deoxyheptonate aldolase | Saccharomyces cerevisiae S288c | modified codon usage for Corynebacterium glutamicum and Saccharomyces cerevisiae | P14 843 | Phospho-2-dehydro-3-deoxyheptonate aldolase | Saccharomyces cerevisiae S288c | modified codon usage for Cg and Sc |
| ScPC A_41 | 134.84 | Q9X5D2 | 3-dehydroquinate synthase | Corynebacterium glutamicum ATCC 13032 | modified codon usage for Cg and Sc | Q9X5D2 | 3-dehydroquinate synthase | Corynebacterium glutamicum ATCC 13032 | modified codon usage for Corynebacterium glutamicum and Saccharomyces cerevisiae | Q9 X5D 2 | 3-dehydroquinate synthase | Corynebacterium glutamicum ATCC 13032 | modified codon usage for Cg and Sc |
| ScPC A_43 | 170.48 | Q8NRS1 | Enolase | Corynebacterium glutamicum ATCC 13032 | modified codon usage for Cg and Sc | Q8NRS1 | Enolase | Corynebacterium glutamicum ATCC 13032 | modified codon usage for Corynebacterium glutamicum and Saccharomyces cerevisiae | Q8 NR S1 | Enolase | Corynebacterium glutamicum ATCC 13032 | modified codon usage for Cg and Sc |
| ScPC A_45 | 79.638 | P08566 | 3-dehydroquinate synthase,3-phosphoshikimate 1-carboxy-vinyltransferase,3-phosphoshikimate 1-carboxyvinyl-transferase,Shikimate kinase (SK),Shikimate 5-dehydrogenase,3-dehydroquinate dehydratase (3-dehydroquinase) | Saccharomyces cerevisiae S288c | modified codon usage for Cg and Sc | P00924 | Enolase | Saccharomyces cerevisiae S288c | modified codon usage for Corynebacterium glutamicum and Saccharomyces cerevisiae | P00 924 | Enolase | Saccharomyces cerevisiae S288c | modified codon usage for Cg and Sc |
| ScPC A_47 | 162.82 | P08566 | 3-dehydroquinate synthase,3-phosphoshikimate 1-carboxy-vinyltransferase,3-phosphoshikimate 1-carboxyvinyl-transferase,Shikimate kinase (SK),Shikimate 5-dehydrogenase,3-dehydroquinate dehydratase (3-dehydroquinase) | Saccharomyces cerevisiae S288c | modified codon usage for Cg and Sc | P32449 | DAHP synthase | Saccharomyces cerevisiae S288c | Corynebacterium glutamicum | P32 449 | DAHP synthase | Saccharomyces cerevisiae S288c | Cg |
| ScPC A_48 | 266.57 | Q9X5D2 | 3-dehydroquinate synthase | Corynebacterium glutamicum ATCC 13032 | modified codon usage for Cg and Sc | P32449 | DAHP synthase | Saccharomyces cerevisiae S288c | Corynebacterium glutamicum | P32 449 | DAHP synthase | Saccharomyces cerevisiae S288c | Cg |
| ScPC A_49 | 240.91 | P532 28 | Transaldolase | Saccharomyces cerevisiae S288c | modified codon usage for Cg and Sc | P00924 | Enolase | Saccharomyces cerevisiae S288c | modified codon usage for Corynebacterium glutamicum and Saccharomyces cerevisiae | P32 449 | DAHP synthase | Saccharomyces cerevisiae S288c | Cg |
| ScPC A_51 | 170.22 | Q9X5D2 | 3-dehydroquinate synthase | Corynebacterium glutamicum ATCC 13032 | modified codon usage for Cg and Sc | Q8NRS1 | Enolase | Corynebacterium glutamicum ATCC 13032 | modified codon usage for Corynebacterium glutamicum and Saccharomyces cerevisiae | O52 377 | 3-dehydroquinate dehydratase (3-dehydroquinase) | Corynebacterium glutamicum ATCC 13032 | modified codon usage for Cg and Sc |
| ScPC A_52 | 174.58 | O52377 | 3-dehydroquinate dehydratase (3-dehydroquinase) | Corynebacterium glutamicum ATCC 13032 | modified codon usage for Cg and Sc | O523 77 | 3-dehydroquinate dehydratase (3-dehydroquinase) | Corynebacterium glutamicum ATCC 13032 | modified codon usage for Corynebacterium glutamicum and Saccharomyces cerevisiae | O52 377 | 3-dehydroquinate dehydratase (3-dehydroquinase) | Corynebacterium glutamicum ATCC 13032 | modified codon usage for Cg and Sc |
| ScPC A_54 | 188.34 | P32449 | Phospho-2-dehydro-3-deoxyheptonate aldolase | Saccharomyces cerevisiae S288c | modified codon usage for Cg and Sc | P324 49 | Phospho-2-dehydro-3-deoxyheptonate aldolase | Saccharomyces cerevisiae S288c | modified codon usage for Corynebacterium glutamicum and Saccharomyces cerevisiae | P32 449 | Phospho-2-dehydro-3-deoxyheptonate aldolase | Saccharomyces cerevisiae S288c | modified codon usage for Cg and Sc |

### REFERENCES

1. Pacheco-Palencia, L.A., S. Mertens-Talcott, and S.T. Talcott, Chemical composition, antioxidant properties, and thermal stability of a phytochemical enriched oil from Acai (Euterpe oleracea Mart.). J Agric Food Chem, 2008. 56(12): p. 4631-6.

What is claimed is:

1. An engineered microbial cell that expresses a non-native 3-dehydroshikimate dehydratase, wherein the engineered microbial cell produces 3,4-dihydroxybenzoic acid.

2. The engineered microbial cell of claim 1, wherein the engineered microbial cell comprises increased activity of one or more upstream 3,4-dihydroxybenzoic acid pathway enzyme(s), said increased activity being increased relative to a control cell.

3. The engineered microbial cell of claim 2, wherein the one or more upstream 3,4-dihydroxybenzoic acid pathway enzyme(s) are selected from the group consisting of an enolase, a transketolase, a transaldolase, phospho-2-dehydro-3-deoxyheptonate aldolase, a 3-deoxy-D-arabino-heptulosonate-7-phosphate (DAHP) synthase, a 3-dehydroquinate synthase, and a 3-dehydroquinate dehydratase.

4. The engineered microbial cell of claim 3, wherein the one or more upstream 3,4-dihydroxybenzoic acid pathway enzyme(s) are selected from the group consisting of an enolase, a transaldolase, a 3-dehydroquinate synthase, and a 3-dehydroquinate dehydratase.

5. The engineered microbial cell of any one of claims 1-4, wherein the engineered microbial cell comprises reduced activity of one or more enzyme(s) that consume one or more 3,4-dihydroxybenzoic acid pathway precursors, said reduced activity being reduced relative to a control cell.

6. The engineered microbial cell of claim 5, wherein the one or more enzyme(s) that consume one or more 3,4-dihydroxybenzoic acid pathway precursors comprise shikimate:NADP+ 3-oxidoreductase.

7. The engineered microbial cell of claim 5 or claim 6, wherein the reduced activity is achieved by replacing a native promoter of a gene for said one or more enzymes with a less active promoter.

8. The engineered microbial cell of any one of claims 1-7, wherein the engineered microbial cell additionally expresses a feedback-deregulated DAHP synthase.

9. The engineered microbial cell of any one of claims 1-8, wherein the engineered microbial cell comprises a fungal cell.

10. The engineered microbial cell of claim 9, wherein the engineered microbial cell comprises a yeast cell.

11. The engineered microbial cell of claim 10, wherein the yeast cell is a cell of the genus Saccharomyces.

12. The engineered microbial cell of claim 11, wherein the yeast cell is a cell of the species cerevisiae.

13. The engineered microbial cell of any one of claims 1-12, wherein the non-native 3-dehydroshikimate dehydratase comprises a 3-dehydroshikimate dehydratase having at least 70% amino acid sequence identity with a 3-dehydroshikimate dehydratase from an organism selected from the group consisting of Neurospora crassa, Corynebacterium glutamicum, Bacillus anthracis, and Gibberella zeae.

14. The engineered microbial cell of claim 13, wherein the non-native 3-dehydroshikimate dehydratase comprises a 3-dehydroshikimate dehydratase having at least 70% amino acid sequence identity with a 3-dehydroshikimate dehydratase from Neurospora crassa.

15. The engineered microbial cell of claim 13, wherein the non-native 3-dehydroshikimate dehydratase comprises a 3-dehydroshikimate dehydratase having at least 70% amino acid sequence identity with a 3-dehydroshikimate dehydratase from Corynebacterium glutamicum.

16. The engineered microbial cell of any one of claims 4 or 9-15, wherein the increased activity of the enolase is achieved by heterologously expressing an enolase.

17. The engineered microbial cell of claim 16, wherein the heterologous enolase comprises an enolase from Saccharomyces cerevisiae.

18. The engineered microbial cell of any one of claims 4 or 9-17, wherein the increased activity of the transaldolase is achieved by heterologously expressing a transaldolase.

19. The engineered microbial cell of claim 18, wherein the heterologous transaldolase comprises a transaldolase from Corynebacterium glutamicum or Saccharomyces cerevisiae.

20. The engineered microbial cell of any one of claims 4 or 9-19, wherein the increased activity of the 3-dehydroquinate synthase is achieved by heterologously expressing a 3-dehydroquinate synthase.

21. The engineered microbial cell of claim 20, wherein the heterologous 3-dehydroquinate synthase comprises a 3-dehydroquinate synthase from Corynebacterium glutamicum or Saccharomyces cerevisiae.

22. The engineered microbial cell of claim 21, wherein the heterologous 3-dehydroquinate synthase comprises a 3-dehydroquinate synthase from Saccharomyces cerevisiae.

23. The engineered microbial cell of claim 22, wherein the heterologous 3-dehydroquinate synthase is from S. cerevisiae 288c (UniProt ID P08566) and comprises SEQ ID NO:6, wherein, the engineered microbial cell also expresses:
a 3-dehydroshikimate dehydratase from Neurospora crassa ATCC 24698 (UniProt ID P07046) comprising SEQ ID NO:1;
a transaldolase from S. cerevisiae 288c (UniProt ID P53228) comprising SEQ ID NO:5; and/or
an enolase from S. cerevisiae 288c (UniProt IDP00924) comprising SEQ ID NO:7.

24. The engineered microbial cell of any one of claims 8, or 9-23, wherein the feedback-deregulated DAHP synthase is a variant of a S. cerevisiae feedback-deregulated DAHP synthase.

25. The engineered microbial cell of claim 24, wherein the feedback-deregulated DAHP synthase is from S. cerevisiae (UniProt ID P32449), comprises amino acid substitution K229L, and comprises SEQ ID NO:3, wherein the engineered microbial cell also expresses:
a 3-dehydroshikimate dehydratase from Neurospora crassa ATCC 24698 (UniProt ID P07046) comprising SEQ ID NO:1;
a 3-dehydroshikimate dehydratase from C. glutamicum ATCC 13032 (UniProt ID O52377) comprising SEQ ID NO:9; and/or
a transaldolase from C. glutamicum ATCC 13032 (UniProt ID Q8NQ64) comprising SEQ ID NO:8.

26. The engineered microbial cell of any one of claims 1-25, wherein, when cultured, the engineered microbial cell produces 3,4-dihydroxybenzoic acid at a level at least 350 mg/L of culture medium.
